(19) United States
(12) Patent Application Publication
(10) Pub. No.: US 2017/0313959 A1
(43) Pub. Date: Nov. 2, 2017

(54) DETERGENTS AND CLEANING AGENTS CONTAINING AT LEAST TWO PROTEASES

(71) Applicant: Henkel AG & Co. KGaA, Duesseldorf (DE)

(72) Inventors: Timothy O'Connell, Landsberg am Lech (DE); Susanne Tondera, Duesseldorf (DE); Thomas Weber, Dormagen (DE)

(73) Assignee: Henkel AG & Co. KGaA, Duesseldorf (DE)

(21) Appl. No.: 15/526,554

(22) PCT Filed: Oct. 28, 2015
(86) PCT No.: PCT/EP2015/074971

§ 371 Date: May 12, 2017

(30) Foreign Application Priority Data
Nov. 14, 2014 (DE) 10 2014 223 296.1

(51) Int. Cl. C11D 3/386; C12N 9/54

(52) U.S. Cl. CPC C11D 3/386; C12N 9/54; C12Y 304/21062

(57) ABSTRACT

An agent and a method for cleaning textiles or hard surfaces are provided herein. In one embodiment, the agent includes a first protease and at least one second protease. The first protease Includes an amino acid sequence that is at least about 80% identical to the amino acid sequence indicated in SEQ ID NO.1. The second protease is any protease that is different from the first protease. In another embodiment, the method includes activating proteolytically the first protease and the at least one second protease.

### CROSS-REFERENCE TO RELATED APPLICATION

This application is a U.S. National-Stage entry under 35 U.S.C. §371 based on International Application No. PCT/EP20151074971, filed Oct. 28, 2015, which was published under PCT Article 21(2) and which claims priority to German Application No. 10 2014 223 296,1, filed Nov. 14, 2014, which are all hereby incorporated hi their entirety by reference.

### TECHNICAL FIELD

This disclosure relates to agent compositions, particularly detergents and cleaning agents, containing a first protease and at least one second protease, and optionally a third or more proteases as well. This disclosure also relates to cleaning methods in which these agents are used, uses of these agents, as well as cleaning methods and uses of these proteases.

### BACKGROUND

Proteases are among the most technically important enzymes. Among these, subtilisin-type proteases (subtilases, subtilopeptidases, EC 3.4.21.62), which are counted among the serine proteases due to their catalytically active amino acids, are of particular importance. They act as nonspecific endopeptidases, that is, they hydrolyze any acid amide bonds on the interior of peptides or proteins. Their pH optimum usually lies well in the alkaline range. An overview of this family can be found, for example, in the article “Subtilases: Subtilisin-like Proteases” by R. Siezen, pages 75-95 in “Subtilisin enzymes,” edited by R, Boll and C. Betzel, New York, 1996. Subtilases are formed naturally by microorganisms; noteworthy is that they include the subtilisins in particular, which are formed and secreted by Bacillus species, as the most important group among the subtilases.

In addition to other enzymes, proteases are established active ingredients of detergents and cleaning agents. They break down protein-containing contaminants on the article to be cleaned. In the most favorable of cases, synergistic effects are produced between the enzymes and the other components of the agent in question. Subtilases occupy a prominent position among the detergent and cleaning agent proteases due to their favorable enzymatic characteristics such as stability or pH optimum. In addition, they are also suitable for a multitude of other technical applications, for example as components of cosmetics or in organic chemical synthesis.

Examples of subtilisin-type proteases that are preferably used in detergents and cleaning agents are subtilisins BPN′ and Carlsberg, protease PB92, subtilisins 147 and 309, alkaline protease from Bacillus lentus, subtilisin DY, and the subtilases, and the enzymes thermitase, proteinase K and proteases TW3 and TW7, which belong to The subtilases but no longer to the subtilisins in the narrower sense. Subtilisin BPN', which originates from Bacillus amyloliquefaciens or B. subtilis, is known from the work of Vasantha et al, (1984) in J. Bacteriol., Volume 159, pp. 811-819 and of J. A. Wells et al. (1983) in Nucleic Acids Research, Volume 11, pp. 7911-7925. Subtilisin BPN′ is used as a reference enzyme of the subtilisins particularly in the context of the numbering of positions. Subtilisin Carlsberg is available in a further developed form under the trade name Alcalase® from Novozymes A/S, Bagsvaerd, Denmark. It is described in the publications of E. L. Smith et al. (1968) in J. Biol. Chem., Volume 243, pp. 2184-2191, and of Jacobs et al. (1985) in Nucl. Acids Res., Band 13, pp. 8913-8926 and formed naturally from Bacillus licheniformis. Protease PB92 is produced naturally by the alkaliphilic bacterium Bacillus nov. spec. 92 and was available under the trade name Maxacal® from Gist-Brocades, Delft, Netherlands. It is described in its original sequence in patent application EP 283075 A2. Subtilisins 147 and 309 are sold by Novozymes under The trade names Esperase® and Savinase®, respectively, They originated from Bacillus strains that were disclosed in the application GB 1243784 A, It is from the protease from Bacillus lentus DSM 5483 (WO 91/02792 A1) that variants are derived that are sold under the designation BLAP® and are described particularly in WO 92/21760 A1, WO 95/23221 A1, WO 02/088340 A2, and WO 03/038082 A2. Subtilisin DY was originally described by Nedkov et al. 1985 in Biol. Chem Hoppe-Seyler, Volume 366, pp. 421-430. Some examples of other proteases that can be used are the enzymes available under the trade names Durazym®, Relase®, Everlase®, Nafizym, Natalase®, Kannase®, and Ovozyme ® from Novozymes, those available under the trade names Purafect®, Purafect®OxP, Purafect® Prime, and Properase® from Genencor, the enzyme Protosol® available from Advanced Biochemicals Ltd., Thane, India, the enzyme available under the trade name Wuxi® from Wuxi Snyder Bioproducts Ltd., China, those available under the trade names Proleather® and Protease P® from Amano Pharmaceuticals Ltd., Nagoya, Japan, and the enzyme available under the name Proteinase K-16 from Kao Corp., Tokyo, Japan.

The proteases used in agents as contemplated herein originate either from microorganisms—for example, from microorganisms of the genera Bacillus, Streptomyces, Humicola, or Psudomonas—and/or they are produced from suitable microorganisms using inherently known methods—for example, by using transgenic expression hosts of the genera Bacillus or using filamentous fungi.

Complex protein contaminants and residues place high demands on the protease contained in the respective agent. For example, contaminants are not completely or satisfactorily removed by a protease contained in the agent. Also, other ingredients of the contaminant can diminish the performance of a protease. In order to improve the performance of agents, particularly detergents and cleaning agents, with respect to their proteolytic activity, attempts are made, for example, to obtain new proteases with advantageous characteristics by removing microorganism-containing specimens from natural habitats, for example, and culturing them under the conditions regarded as being appropriate—in alkaline medium, for instance. It is also possible to optimize proteases by employing inherently known mutagenesis methods for use in the respective agents, particularly detergents and cleaning agents. These include point mutagenesis, deletion, insertion, or fusion with other proteins or protein segments, or other, particularly chemical, modifications. For example, the surface charges and/or the isoelectric point of the molecules and hence their interactions with the substrate are altered as a result, or the stability of the proteases in question—and hence their effectiveness—is increased. However, the possibilities described here for the sake of example for further development of the proteases that can be used in corresponding agents have the drawback that they are complex and time-consuming.

With regard to the use of proteases in detergents and cleaning agents, it is known that, in order to improve the detergent and cleaning performance, proteases can be used together with other enzymes, such as amylases, cellulases, hemicellulases, mannanases, β-glucosidases, oxidases, oxidoreductases, or lipases. The use of proteases in detergents in combination with other active substances such as bleaching agents or soil-release agents is also known to a person skilled in the art, Furthermore, it is known from the prior art that commensurate agents, particularly detergents and cleaning agents, can contain several proteases. For instance, international patent application WO 03/054185 A1 discloses that the alkaline protease from Bacillus gibsonli (DSM 14391) can be used in a corresponding agent together with other proteases. However, like the other prior art, this application does not give any indication of the specific proteases with which this enzyme can be advantageously combined in order to achieve a synergistic coaction of the proteases in a corresponding agent.

### BRIEF SUMMARY

An agent and a method for cleaning textiles or hard surfaces are provided herein. In one embodiment, the agent includes a first protease and at least one second protease. The first protease includes an amino acid sequence that is at least about 80% identical to the amino acid sequence indicated in SEQ ID NO.1. The second protease is any protease that is different from the first protease. In another embodiment, the method includes activating proteolytically the first protease and the at least one second protease.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it was found that the performance of agents, particularly of detergents and cleaning agents, is improved significantly with respect to their proteolytic activity if mixtures of at least two specific proteases having a different spectrum of activity and/or different sequence are used in these agents. A synergistic effect is produced with respect to the coaction of these specific, different proteases; that is, better performance is achieved in comparison to the individual performance of the respective protease in a one-component system (i.e., agents containing only these proteases) and in comparison to the sum of the individual performance levels of the proteases, i.e., the sum of two one-component systems, for example. The selected combination of specific proteases thus represents another possibility for improving the performance of agents, particularly detergents and cleaning agents, in terms of their proteolytic activity.

It is therefore the object of the present disclosure to provide agents having improved proteolytic activity.

Associated with this is another object of the present disclosure, namely to provide agents having improved detergent and cleaning performance, particularly at low temperatures, preferably at about 20° C., in relation to at least one protein-containing contaminant, preferably in relation to several protein-containing contaminants.

Another special object of the present disclosure is to provide agents having an at least equal, preferably an improved detergent and cleaning performance in relation to at least one protein-containing contaminant, preferably in relation to several protein-containing contaminants, but having a reduced protease content.

This object is achieved as contemplated herein through the provision of agents which contain mixtures of specially selected proteases with different spectra of activity and/or different sequences. In these agents as contemplated herein, the proteases contained and specially selected for this purpose develop a synergistic cleaning performance. Consequently, the agents achieve better removal of protein-containing contaminants than agents containing either only one protease or agents containing two proteases but whose cleaning performance proves to be merely commensurate with the addition of the respective individual contributions of the two proteases contained. The selected combination of such proteases therefore constitutes an essential aspect of the disclosure and has a synergistic effect in agents as contemplated herein in terms of the removal of protein-containing residues or contaminants.

One object of the disclosure is therefore agents containing a first protease and at least one second protease which are characterized in that
a) the first protease comprises an amino acid sequence that is at least about 80% identical and, in order of increasing preference, at least about 82.5%, about 85%, about 87.5%, about 90%, about 91%, about 92%, about 93%, about 94%, about 95%, about 96%, about 97%, about 98%, about 99%, about 99.5% identical, and very especially preferably 100% identical to the amino acid sequence indicated in SEQ ID NO.1 and
b) the second protease is any protease That is different from the first protease.

Preferably, the second protease is selected from among proteases comprising an amino acid sequence that is at least 80% and, in order of increasing preference, at least about 82.5%, about 85%, about 87.5%, about 90%, about 91%, about 92%, about 93%, about 94%, about 95%, about 96%, about 97%, about 98%, about 99%, about 99.5%, and very especially preferably 100% identical to one of the amino acid sequences indicated in SEQ ID NO.2 to SEQ ID NO.7, particularly from among proteases comprising an amino acid sequence that is at least about 80% identical and, in order of increasing preference, at least about 82.5%, about 85%, about 87.5%, about 90%. about 91%, about 92%. about 93%. about 94%, about 95%, about 96%, about 97%, about 98%, about 99%, about 99.5% Identical, and very especially preferably 100% identical to one of The amino acid sequences indicated in SEQ ID NO.6 or SEQ ID NO.7.

In addition to the first and the second protease, the agent as contemplated herein can also contain one or more other proteases, which can be any proteases that are different from the first and the second protease but are preferably selected from among proteases comprising an amino acid sequence that is at least about 80% and, in order of increasing preference, at least about 82.5%, about 85%, about 87.5%, about 90%. about 91%, about 92%, about 93%, about 94%. about 95%, about 96%, about 97%, about 98%, about 99%, about 99.5%, and very especially preferably 100% identical to one of the amino acid sequences indicated in SEQ ID NO.2 to SEQ ID NO.7.

Agents as contemplated herein contain the above-described at least two proteases particularly in total quantities from about 0.05 to about 5 percent by weight (wt %), preferably about 0,05 to about 2 wt %, with respect to the total quantity of the agent.

With respect to active protein, the agents as contemplated herein contain the above-described at least two proteases particularly in total quantities from about 0.05 to about 15 wt %, preferably about 0.05 to about 10 wt %.

Another object of the disclosure is therefore an enzyme mixture containing a first protease and at least one second protease which are characterized in that
a) the first protease comprises an amino acid sequence that is at least 80% identical and, in order of increasing preference, at least about 82.5%, about 85%, about 87.5%, about 90%, about 91%, about 92%, about 93%, about 94%, about 95%, about 96%, about 97%, about 98%, about 99%, about 99.5% Identical, and very especially preferably 100% identical to the amino acid sequence indicated irl SEQ ID NO.1 and
b) the second protease is any protease that is different from the first protease.

Preferably, the second protease is selected from among proteases comprising an amino acid sequence that is at least about 80% and, in order of increasing preference, at least about 82.5%, about 85%, about 87,5%, about 90%, about 91%, about 92%, about 93%, about 94%, about 95%, about 96%, about 97%, about 98%, about 99%, about 99.5%, and very especially preferably 100% identical to one of the amino acid sequences indicated in SEQ ID NO.2 to SEQ ID NO.7, particularly from among proteases comprising an amino acid sequence that is at least about 80% identical and, in order of Increasing preference, at least about 82.5%, about 85%, about 87.5%, about 90%, about 91%, about 92%, about 93%, about 94%, about 95%, about 96%, about 97%, about 98%, about 99%, about 99.5% identical, and very especially preferably 100% identical to one of the amino acid sequences indicated in SEQ ID NO.6 or SEQ ID NO.7.

In addition to the first and the second protease, the enzyme mixture as contemplated herein can also contain other proteases, which can be any proteases that are different from the first and the second protease but are preferably selected from among proteases comprising an amino acid sequence that is at least about 80% and, in order of increasing preference, at least about 82.5%, about 85%, about 87.5%, about 90%, about 91%, about 92%, about 93%, about 94%, about 95%, about 96%, about 97%, about 98%, about 99%, about 99.5%, and very especially preferably 100% identical to one of the amino acid sequences indicated in SEQ ID NO.2 to SEQ ID NO.7.

All facts, subject matter and embodiments that are described for detergents or cleaning agents as contemplated herein are applicable analogously to the cited enzyme mixtures as well. Reference is Therefore made here expressly to the disclosure in the corresponding location, with This disclosure also applying to the abovementioned enzyme mixtures as contemplated herein.

Surprisingly, the inventive combination of a protease whose amino acid sequence corresponds to the amino acid sequence indicated in SEQ ID NO.1 or is at least 80% identical thereto with at least one other protease in an agent as contemplated herein brings about improved synergistic cleaning performance. The synergistic effect is based on the fact that the effects of the mutually different proteases complement one another such that an enhanced proteolytic effect is achieved overall.

Also surprisingly. it was found that this improved effect occurs particularly at low temperatures, preferably in the temperature range from about 10 ° C. to about 30 ° C., especially preferably at about 20 ‘C.

For example, the improved effect can be enabled as a result of the fact that the proteolytic cleaving of peptide bonds is made more accessible by the first protease substrate for the second protease and, as a result of the proteolytic cleaving of the peptide bonds by the second protease, other substrates are, in turn, made accessible for the first protease. Particularly in the case of complex, protein-containing substrates, the accessibility of a cleavage site is often a limiting factor for a protease. A different spectrum of activity of the two proteases therefore manifests itself in a different substrate specificity, for example. likewise, a different spectrum of activity can be brought about by a different proteolytic activity under defined conditions, particularly with respect to temperature, pH value, ionic strength, stability in relation to oxidizing compounds (bleaching agents, for example), etc.

Synergism as contemplated herein therefore occurs when the first protease differs from the second protease and from other proteases that may be present. These differences manifest themselves in the amino acid sequence of the respective proteolytically active enzyme.

In terms of the present application, an enzyme is to be understood as a protein that carries out a certain biocatalytic function, in terms of the present application, a protease is understood as being an enzyme that catalyzes the hydrolysis of peptide bonds and is thus capable of cleaving peptides or proteins,

In terms of the present application, a protein is a polypeptide composed of the natural amino acids and has largely linear structure that is usually three-dimensional for the purpose of carrying out its function. A peptide consists of amino acids that are covalently bonded to one another via peptide bonds. The term polypeptide makes the fact dear in this regard that this peptide chain generally consists of many amino acids that are interconnected by peptide bonds. Amino acids can be present in an L and a D configuration, with the amino acids of which proteins are made being present in the L configuration. They are referred to as proteinogenic amino acids. in the present application, the proteinogenic, naturally occurring L-amino acids are referred to using the customary international 1- and 3-letter codes. Numerous proteins are formed as so-called preproteins, i.e., together with a signal peptide. This, in turn, is to be understood as the N-terminal portion of the protein, whose function usually consists in discharging the formed protein from the producing cell into the periplasma or the surrounding medium and/or ensuring the correct folding thereof. The signal peptide is then cleaved from the rest of the protein by a signal peptidase under natural conditions, so that it can carry out its actual catalytic activity without the initially present N-terminal amino acids. Pro-proteins are inactive precursors of proteins. The precursors of these with signal sequence are referred to as pre-pro-proteins. Due to their enzymatic activity, the mature peptides, that is, enzymes that have been processed after their production, are preferred over the preproteins for technical applications.

The proteins can be modified by the cells producing them after the production of the polypeptide chain, for example through the linking of sugar molecules, formylation, amination, etc. Such modifications are referred to as post-translational modifications. These post-translational modifications can but do not necessarily have an influence on the function of the protein.

Through comparison with known enzymes that are filed in generally accessible databases, for example, the enzymatic activity of a given enzyme can be inferred from the amino acid or nucleotide sequence, These can be modified qualitatively or quantitatively by other regions of the protein that are not involved in the actual reaction. This may relate to the enzyme stability, the activity, the reaction conditions, or the substrate specificity.

Such a comparison is done by correlating similar sequences in the nucleotide or amino acid sequences of the respective proteins. This is called homologation. A tabular correlation of the respective positions is referred to as alignment. During the analysis of nucleotide sequence, both complementary strands and all three possible respective reading frames as well as The degeneracy of the genetic code and the organism-specific use of the codon (codon usage) must also be taken into account. Alignments are now performed using computer programs with algorithms such as FASTA or BLAST; this procedure is described, for example, by D. J, Lipman and W. R. Pearson (1985) in Science, Volume 227, pp. 1435-1441. All of the sequence comparisons and determinations of homology and/or identity values performed in the present application are carried out using the computer program Vector NTI® Suite 7.0, available from InforMax, Inc., Bethesda, USA, using the preset default parameters.

A compilation of all of the corresponding positions in the compared sequences is referred to as a consensus sequence. Such a comparison also provides information regarding the similarity or homology of the compared sequences in relation to one another. This is expressed in percent identity, That is, the proportion of identical nucleotides or amino acid residues therein or in an alignment of mutually corresponding positions. A broader concept of homology includes the conserved amino acid exchanges in this value. One then speaks of percent similarity, Such characterizations can be made about entire proteins or genes or only about individual regions.

Homologous regions of different proteins are defined by matches in the amino acid sequence. These can also be characterized by identical function. It can range to complete identity in the similest regions, so-called boxes, which comprise only a few amino acids and usually carry out functions that are essential for the overall activity. The functions of the homologous regions are to be understood as being the smallest subfunctions of the function carried out by the overall protein, such as the formation of individual hydrogen bonds for the complexation of a substrate or transition complex, for example. Proteases and enzymes in general can be further developed by various methods, such as targeted genetic alteration by employing mutagenesis methods, and optimized for specific applications or with respect to special characteristics, such as catalytic activity, stability, etc.

Furthermore, it is generally known from the prior art that advantageous characteristics of individual mutations, e.g., individual point mutations, can complement one another. A protease that is already optimized with respect to certain characteristics for example, with respect to its stability in relation to surfactants or other components—can be additionally further developed as contemplated herein.

Fragments are understood as being all proteins or peptides that are smaller than natural proteins and can be obtained synthetically, for example. They can be correlated with the respective complete proteins on the basis of their amino acid sequences. For example, they can take on the same structures or carry out proteolytic activities or subactivities, such as the complexation of a substrate. Fragments and deletion variants of starting proteins are similar in principle; while fragments tend to be smaller parts, the deletion mutants tend to lack only small regions and therefore only individual subfunctions.

In terms of the present application, chimeric or hybrid proteins are to be understood as proteins whose sequence includes the sequences or subsequences of at least two starting proteins. The starting proteins can originate from different organisms or from the same one. Chimeric or hybrid proteins can be obtained through recombination mutagenesis, for example. The purpose of such recombination can, for example, be to bring about or modify a specific enzymatic function with the aid of the fused protein segment. In terms of the present disclosure, it is unimportant in this regard whether such a chimeric protein consists of a single polypeptide chain or several subunits to which different functions can be distributed.

Proteins obtained through insertion mutation are to be understood as those variants which have been obtained through the insertion of a protein fragment into the starting sequences. Due to their basic similarity, they are grouped with the chimeric proteins. They differ from them only in the proportion of the unaltered protein segment to the size of the overall protein. In such insertion-mutated proteins, the proportion of foreign protein is lower than in chimeric proteins.

Inversion mutagenesis, that is, a partial reversal of a sequence, can be regarded as being a special form both of deletion and of insertion. The same applies to a regrouping of different moieties that differs from the original amino acid sequence. It can be regarded as a deletion variant, an insertion variant, and as a shuffling variant of the original protein.

In terms of the present application, derivatives are to be understood as proteins whose amino acid chain has been chemically modified. Such derivatizations can take place biologically, for example, in connection with protein biosynthesis by the host cell. Methods from molecular biology can be used for this purpose. They can also be performed chemically, however, for instance through the chemical conversion of a side chain of an amino acid or through covalent bonding of another compound to the protein. Such a compound can also be other proteins, for example, that are joined by employing bifunctional chemical bonds to proteins as contemplated herein, for example. Such modifications can influence the substrate specificity or the strength of the bond to the substrate, for example, or bring about a temporary blocking of the enzymatic activity if the coupled substance is an inhibitor. This can be expedient during a period of storage, for example. Likewise, derivatization is to be understood as covalent bonding to a macromolecular carrier, as well as noncovalent inclusion in suitable macromlecular cage structures. In another embodiment as contemplated herein, the agent is therefore characterized in that the first protease is present in the agent as a fragment, deletion variant, chimeric protein, or derivative and/or the second protease is present in the agent as a fragment, deletion variant, chimeric protein, or derivative, with the first and the second protease still being catalytically active.

In terms of the present disclosure, insofar as they need not be addressed explicitly as such, all enzymes, proteins, fragments, chimeric proteins, and derivatives are grouped under the collective term “protein.”

Agents as contemplated herein comprise all types of agent, particularly mixtures, formulations, solutions, etc., whose usability is improved through the addition of the above-described proteases. Depending on the area of application, they can be solid mixtures such as powders with freeze-dried or encapsulated proteins, or del-type or liquid agents. Preferred formulations contain buffer substances, stabilizers, reaction partners, and/or cofactors of the proteases and/or other ingredients that are synergistic with the proteases. In particular, they are to be understood as agents for the areas of application set out further below. Other areas of application follow from the prior art and are described, for example, in the handbook “Industrial enyzmes and their applications” by H. Uhlig, Wiley, New York, 1998.

In preferred embodiments as contemplated herein, an agent is characterized in that it is a detergent, handwashing composition, dishwashing detergent, dishwashing liquid, dishwasher detergent, cleaning agent, denture or contact lens care product, rinse aid, disinfectant, cosmetic agent, pharmaceutical agent, or a product for treating filter media, textiles, pelts, papers, furs, or leather, particularly a laundry detergent or a dishwashing detergent.

This object of the disclosure includes all conceivable types of detergent and cleaning agent, as well as concentrates and agents to be used in undiluted form, for use on a commercial scale, in the washing machine, or for hand washing or cleaning. These include, for example, detergents for textiles, carpeting, or natural fibers, for which the term “detergent” is used according to the present disclosure. They also include dishwashing detergents for dishwashers or hand dishwashing liquids or cleaners for hard surfaces such as metal, glass, porcelain, ceramic, tile, stone, painted surfaces, plastics, wood, or leather, for example; according to the present disclosure, the term “cleaning agent” is used for these.

An agent as contemplated herein can be an agent for large-scale consumers or technical users and a product for private consumers, with all types of detergent and cleaning agent established in the prior art also constituting embodiments of the present disclosure.

The detergents or cleaning agents as contemplated herein, which can come particularly in the form of powdered solids, in the form of compressed particles, as homogeneous solutions, or suspensions, can in principle contain, in addition to the active substance used as contemplated herein—an inventive combination of proteases all ingredients that are known and commonly contained in such agents, with at least one other ingredient being present in the agent. The agents as contemplated herein can particularly contain builder substances, surface-active surfactants, bleaching agents based on organic and/or inorganic peroxide compounds, bleach activators, water-miscible organic solvents, enzymes, sequestering agents, electrolytes, pH regulators, and other adjuvants such as optical brighteners, anti-redeposition agents, foam control agents, and colorants and perfumes, as well as combinations thereof. In particular, combining the combination of proteases with one or more other ingredients of the agents proves advantageous, since such an agent has improved cleaning performance due to the resulting synergisms, particularly between a protease or combination of proteases and the other ingredient. This means that the agent brings about improved removal of contaminants, such as protein-containing contaminants, for example, in comparison to an agent that contains either only one of the two components or even in comparison to the expected cleaning performance of an agent with both components based on the mere addition of the respective individual contributions of the two components to the cleaning performance of the agent. Such synergism is achieved particularly through the combination of an inventive combination of proteases with one of the surfactants and/or builder substances and/or bleaching agents described below.

The agents as contemplated herein can contain a surfactant or several surfactants, with anionic surfactants, nonionic surfactants, and mixtures thereof, as well as cationic, zwitterionic, and amphoteric surfactants being particularly worthy of consideration.

Suitable nonionic surfactants are particularly alkyl glycosides and ethoxylation and/or propoxylation products of alkyl glycosides or linear or branched alcohols each with 12 to 18 C atoms in the alkyl portion and 3 to 20, preferably 4 to 10 alkyl ether groups. Moreover, corresponding ethoxyiation and/or propoxylation products of N-alkyl amines, vicinal diols, fatty acid esters, and fatty acid amides that correspond with respect to the alkyl portion to the cited long-chain alcohol derivatives, as well as of alkyl phenols with 5 to 12 C atoms in the alkyl residue.

Nonionic surfactants that are preferably used are alkoxylated, preferably ethoxylated, particularly primary alcohols with preferably 8 to 18 C atom and, on average, about 1 to about 12 mols of ethylene oxide (EO) per mol of alcohol in which the alcohol residue can be linear or preferably methyl-branched in the 2 position, or it can contain linear and methyl-branched residues in admixture, as are usually present in oxa-alcohol residues. In particular, however, alcohol ethoxylates with linear residues from alcohols of native origin with 12 to 18 C atoms, e.g., from coconut, palm, tallow fat, or oleyl alcohol, and 2 to 8 EO per mol of alcohol on average are preferred. Examples of preferred ethoxylated alcohols are C₁₂-C₁₄ alcohols with 3 EO or 4 EO, C₉-C₁₁ alcohols with 7 EO, C₁₃-C₁₅ alcohols with 3 EO, 5 EO, 7 EO or 8 EO, C₁₂-C₁₈ alcohols with 3 EO, 5 EO or 7 EO and mixtures thereof, such as mixtures of C₁₂-C₁₄ alcohol with 3 EO and C₁₂-C₁₈ alcohol with 7 EO. The indicated degrees of ethoxylation represent statistical averages that can be a whole number or a fraction for a given product. Preferred alcohol ethoxylates have a narrowed homolog distribution (narrow range ethoxylates, NRE). In addition to these nonionic surfactants, fatty alcohols with more than 12 EO can also be used. Examples of these are (tallow) fatty alcohols with 14 EO, 16 EO, 20 EO, 25 EO, 30 EO or 40 EO. Extremely low-foaming compounds are usually used particularly in agents for use in mechanical processes. These include the preferred C₁₂-C₁₈ alkyl polyethylene glycol polypropylene glycol ethers, each with up to 9 mols of ethylene oxide and propylene oxide units in the molecule. Other known low-foaming nonionic surfactants can also be used, however, such as C₁₂-C₁₈ alkyl polyethylene glycol polybutylene glycol ethers, each with up to 8 mols of ethylene oxide and butylene oxide units in the molecule as well as end-capped alkyl polyalkylene glycol mixed ethers. The hydroxyl group-containing alkoxylated alcohols such as those described in European patent application EP 0 300 305—so-called hydroxy mixed ethers—are also especially preferred. The nonionic surfactants used also include alkyl glycosides of the general formula RO(G)x, in which R stands for a primary straight-chain or methyl-branched aliphatic residue, particularly one that is methyl-branched in the 2 position, with 8 to 22, preferably 12 to ‘18 C atoms and G stands for a glycose unit with 5 or 6 C atoms, preferably for glucose. The degree of oligomerization x, which indicates the distribution of monoglycosides and oligoglycosides, is any number—which can also take on fractional values as an analytically determined value—between about 1 and about 10; preferably, x is from about 1.2 to about 1.4. Likewise suitable are polyhydroxy fatty acid amides of formula (III), in which R CO stands for an aliphatic acyl residue with 6 to 22 carbon atoms, R² stands for hydrogen, an alkyl or hydroxyalkyl residue with 1 to 4 carbon atoms and [Z] stands for a linear or branched polyhydroxyalkyl residue with 3 to 10 carbon atoms and 3 to 10 hydroxyl groups:

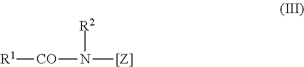

Preferably, the polyhydroxy fatty acid amides are derived from reducing sugars with 5 or 6 carbon atoms, particularly from glucose. The group of the polyhydroxy fatty acid amides also includes compounds of formula (IV).

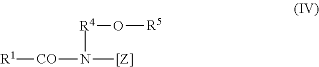

in which R³ stands for a linear or branched alkyl or alkenyl residue with 7 to 12 carbon atoms, R⁴ stands for a linear, branched, or cyclic alkylene residue or an arylene residue with 2 to 8 carbon atoms and R⁵ stands for a linear, branched, or cyclic alkyl residue or an aryl residue or an oxy-alkyl residue with 1 to 8 carbon atoms, with C₁-C₄ alkyl or phenyl residues being preferred, and [Z] stands for a linear polyhydroxyalkyl residue, whose alkyl chain is substituted with at least two hydroxyl groups, or alkoxylated, preferably ethoxylated or propoxylated derivatives of this residue. Here, too, [Z] is preferably obtained through the reductive amination of a sugar such as glucose, fructose, maltose, lactose, galactose, mannose, or xylose. The N-alkoxy- or N-aryloxy-substituted compounds can then be converted into the desired polyhydroxy fatty acid amides through conversion with fatty acid methyl esters in the presence of an alkoxide as a catalyst. Another class of preferred nonionic surfactants, which are used either as the sole nonionic surfactant or in combination with other nonionic surfactants, particularly together with alkoxylated fatty alcohols and/or alkyl glycosides, are alkoxylated, preferably ethoxylated or ethoxylated and propoxylated fatty acid alkyl esters, preferably with 1 to 4 carbon atoms in the alkyl chain, particularly fatty acid methyl esters. Nonionic surfactants of the type of the aminoxides, for example N-cocoalkyl-N,N-dimethylamine oxide and N-tallow alkyl-N,N-dihydroxyethylamine oxide, and of the fatty acid alkanolamides can also be suitable. The quantity of these nonionic surfactants is preferably no more than that of the ethoxylated fatty alcohols, particularly no more than half thereof. So-called gemini surfactants merit consideration as other surfactants. These are generally regarded as being compounds having two hydrophilic groups per molecule. These groups are generally separated from one another by a so-called “spacer.” This spacer is generally a carbon chain, which should be long enough so that the hydrophilic groups are spaced apart sufficiently that they can act independently of one another. Such surfactants are generally characterized by an unusually low critical micelle concentration and the ability to strongly reduce the surface tension of water. In exceptional cases, the term “gemini surfactants” is understood as referring not only to such “dimeric” surfactants but also to “trimeric” surfactants as well. Some examples of suitable gemini surfactants are sulfated hydroxy mixed ethers or dimer alcohols-bis- and trimeralcohol-tris-sulfates and ether sulfates. End-capped dimeric and trimeric mixed ethers are particularly characterized by their bi- and multifunctionality. For instance, the cited end-capped surfactants have good wetting characteristics and yet are low-foaming, so they are particularly suitable for use in mechanical washing or cleaning processes. Gemini polyhydroxy fatty acid amides or poly-polyhydroxy fatty acid amides can also be used, however. Sulfuric acid mortoesters of straight-chain or branched C₇-C₂₁ alcohols ethoxylated with about 1 to about 6 mols of ethylene oxide, such as 2-methyl-branched C₉-C₁₁ alcohols with about 3.5 mols of ethylene oxide (EO) on average or C₁₂-C₁₈ fatty alcohols with 1 to 4 EO are also suitable. The preferred anionic surfactants also include the salts of alkyl sulfosuccinic acid, which are also referred to as sulfosuccinates or as sulfosuccinic acid esters, and the monoesters and/or diesters of sulfosuccinic acid with alcohols, preferably fatty alcohols and particularly ethoxylated fatty alcohols. Preferred sulfosuccinates contain C₈ to C₁₈ fatty alcohol residues or mixtures thereof. Particularly preferred sulfosuccinates contain a fatty alcohol residue that is derived from ethoxylated fatty alcohols, which, per se, represent nonionic surfactants. In turn, sulfosuccinates whose fatty alcohol residues are derived from ethoxylated fatty alcohols with narrowed homolog distribution are especially preferred. Likewise, it is also possible to use alk(en)yl succinic acid with preferably 8 to 18 carbon atoms in the alk(en)yl chain or salts thereof. Other anionic surfactants meriting consideration are fatty acid derivatives of amino acids, for example of N-methyltaurine (taurides) and/or of N-methylglycine (sarcosides). Among these, the sarcosides or sarcosinates, particularly sarcosinates of higher and optionally mono- or polyunsaturated fatty acids such as oleyl sarcosinate are particularly preferred. Soaps in particular merit consideration as other anionic surfactants. Particularly, saturated fatty acid soaps such as the salts of lauric acid, myristic acid, palmitic acid, stearic acid, hydrated erucic acid, and behenic acid, as well as soap mixtures derived particularly from natural fatty acids, such as coconut, palm kernel, or tallow fatty acids, are suitable. The known alkenyl succinic acid salts can also be used together with these soaps or in replacement of soaps.

The anionic surfactants, including the soaps, can be present in the form of their sodium, potassium, or ammonium salts as well as soluble salts of organic bases, such as mono-, di-, or triethanolamine. Preferably, the anionic surfactants are present in the form of their sodium or potassium salts, particularly in the form of the sodium salts.

Surfactants are contained in agents as contemplated herein in proportions from preferably about 5 wt % to about 50 wt %, particularly from about 8 wt % to about 30 wt %,

An agent as contemplated herein preferably contains at least one water-soluble and/or water-insoluble, organic and/or inorganic builder. The water-soluble organic builder substances include polycarboxylic acids, particularly citric acids and saccharic acids, monomeric and polymeric aminopolycarboxylic acids, particularly methyl glycine diacetic acid, nitrilotriacetic acid and ethylenediaminetetraacetic acid, as well as polyaspartic acid, polyphosphonic adds, particularly aminotris(methylene phosphonic acid), ethylenediamine tetrakis(methylene phosphonic acid) and 1-hydroxyethane-1, 1-diphosphonic acid, polymeric hydroxy compounds such as dextrin, as well as polymeric (poly)carboxylic acids, particularly the polycarboxylates, polymeric acrylic acids, methacrylic acids, maleic acids and mixed polymers thereof obtainable through the oxidation of polysaccharides or dextrins, which can also contain small proportions of polymerizable substances without carboxylic acid functionality polymerized in. The relative molecular mass of the homopolymers of unsaturated carboxylic acids generally lies between about 3,000 and about 200,000, and that of the copolymers generally lies between about 2,000 and about 200,000, preferably about 30,000 and about 120,000, each with respect to the free acid. An especially preferred acrylic acid-maleic acid copolymer has a relative molecular mass from about 30,000 to about 100,000, Commercially available products include Sokalan® CP 5, CP 10, and PA 30 from BASF.

Suitable albeit less preferred compounds of this class are copolymers of acrylic acid or methacrylic acid with vinyl ethers, such as vinyl methyl ethers, vinyl esters, ethylene, propylene, and styrene, in which the proportion of acid is at least about 50 wt %. Terpolymers containing, as monomers, two unsaturated acids and/or salts thereof and, as a third monomer, vinyl alcohol and/or an esterified vinyl alcohol or a carbohydrate can also be used as water-soluble organic builder substances. The first acidic monomer or salt thereof is derived from a monoethylenically unsaturated C3-C8 carboxylic acid and preferably from a C3C-4 monocarboxylic acid, particularly from (meth)acrylic acid. The second acidic monomer or salt thereof can be a derivative of a C4-C8 dicarboxylic acid, with maleic acid being especially preferred, and/or a derivative of an allyl sulfonic acid that is substituted in the 2 position with an alkyl or aryl residue. Such polymers generally have a relative molecular mass between about 1,000 and about 200,000. Other preferred copolymers are those which preferably have acrolein and acrylic acid/acrylic acid salts or vinyl acetate as monomers. Particularly for the manufacture of liquid agents, the organic builder substances can be used in the form of aqueous solutions, preferably in the form of about 30 to about 50 wt % aqueous solutions. All of the cited acids are generally used in the form of their water-soluble salts, particularly their alkali salts.

Such organic builder substances can be contained as desired in quantities of up to about 40 wt %, particularly up to about 25 wt %, and preferably from about 1 wt % to about 8 wt.%. Quantities near the cited upper limit are preferably used in pasty or liquid, particularly water-containing, agents as contemplated herein.

Meriting consideration as water-soluble inorganic builder materials are, particularly, alkali silicates, alkali carbonates, and alkali phosphates, which can be present in the form of their alkaline, neutral, or acidic sodium or potassium salts. Examples of these are trisodium phosphate, tetrasodium diphosphate, disodium hydrogen phosphate, pentasodium triphosphate, so-called sodium hexametaphosphate, oligomeric trisodium phosphate with degrees of oligomerization from about 5 to about 1000, particularly about 5 to about 50, as well as the corresponding potassium salts and mixtures of sodium and potassium salts. Crystalline or amorphous alkali aluminosilicates are particularly used as water-insoluble, water-dispersible inorganic builder materials in quantities of up to about 50 wt %, preferably of no more than about 40 wt %, and in liquid agents particularly from about 1 wt % to about 5 wt %. Among these, the crystalline sodium aluminosilicates in detergent quality, particularly zeolite A, P, and X where appropriate, either alone or in mixtures, for example in the form of a co-crystallisate of zeolites A and X (Vegobond® AX, a commercial product from Condea Augusta S.p.A.), are preferred. Quantities near the cited upper limit are preferably used in solid, particulate agents. Suitable aluminosilicates have particularly no particles with a grain size of over about 30 μm and preferably consist of at least about 80 wt % of particles with a grain size of below about 10 μm. Their calcium binding capacity, which can be determined according to the specifications of German patent DE 24 12 837, generally lies in the range from about 100 to about 200 mg CaO per gram.

Suitable substitutes or partial substitutes for the cited aluminosilicate are crystalline alkali silicates, which can be present alone or in admixture with amorphous silicates. The alkali silicates that can be used in the agents as contemplated herein as builder materials preferably have a molar ratio of alkali oxide to SiO2 of less than about 0.95, particularly from about 1:1.1 to about 1:12, and can be present in amorphous or crystalline form. Preferred alkali silicate are the sodium silicates, particularly the amorphous sodium silicates, with a molar ratio of Na2O:SiO2 from about 1:2 to about 1:2,8. Crystalline layered silicates of the general formula Na2SixO2x+1 y H2O are preferably used as crystalline silicates that can be present alone or in admixture with amorphous silicates, where x, the so-called module, is a number from about 1.9 to about 22, particularly about 1.9 to about 4, and y is a number from 0 to 33 and preferred values for x are 2, 3, or 4. Preferred crystalline layered silicates are those in which x assumes the values 2 or 3 in the cited general formula. In particular, both β- and δ-sodium disilicates (Na2Si2O5.y H2O) are preferred. Practically water-free crystalline alkali silicates of the abovementioned general formula produced from amorphous alkali silicates, where x refers to a number from about 1.9 to about 2.1, can also be used in agents as contemplated herein. In another preferred embodiment of agents as contemplated herein, a crystalline sodium layered silicate with a module from 2 to 3 such as can be produced from sand and soda is used. Crystalline sodium silicates with a module in the range from about 1.9 to about 3.5 are used in another preferred embodiment of agents as contemplated herein. Crystalline layered silicates of formula (I) indicated above are sold by Clariant GmbH under the trade name Na—SKS, for example Na—SKS-1 (Na2Si22O45.xH2O, kenyaite), Na—SKS-2 (Na2Si14O29.xH2O, magadiite), Na—SKS-3 (Na2Si8O17.xH2O) or Na—SKS-4 (Na2Si4O9.xH2O, makatite). Among these, Na—SKS-5 (α-Na2Si2O5), Na—SKS-7 (β-Na2Si2O5, natrosilite), Na—SKS-9 (NaHSi2O5.3H2O), Na—SKS-10 (NaHSi2O5.3H2O, kanemite), Na—SKS-11 (t-Na2Si2O5), and Na—SKS-13 (NaHSi2O5), but particularly Na—SKS-6 (δ-Na2Si2O5). In a preferred embodiment of agents as contemplated herein, a granular compound of crystalline layered silicate and citrate, of crystalline layered silicate and the abovementioned (co)polymeric polycarboxylic acid, or of alkali silicate and alkali carbonate is used, which is commercially available under the name Nabione® 15, Builder substances are preferably contained in the agents as contemplated herein in quantities of up to about 75 wt %, particularly about 5 wt % to about 50 wt %.

As peroxide compounds that are suitable for use in agents as contemplated herein, the following merit particular consideration: organic peroxides or peroxide salts of organic acids, such as phthalimidopercaproic acid, perbenzoic acid, or salts of diperdodecane diacid, hydrogen peroxide, and inorganic salts that give off hydrogen peroxide under washing conditions, which include perborate, percarbonate, persilicate, and/or persulfate, such as caroate. If solid peroxide compounds are to be used, they can be used in the form of powders or granulates, which can in principle also be coated in a known manner. If an agent as contemplated herein contains peroxide compounds, they are present in quantities of preferably up to about 50 wt %, particularly from about 5 wt % to about 30 wt %, The addition of small quantities of known bleaching stabilizers such as phosphonates, borates, or metaborates and metasilicates, as well as magnesium salts such as magnesium sulfate, can be expedient.

Compounds which, under perhydrolysis conditions, yield aliphatic peroxocarboxylic acids with preferably 1 to 10 C atoms, particularly 2 to 4 C atoms, and/or optionally substituted perbenzoic acid, can be used as bleach activators. Substances that carry the O and/or N-acyl group of the cited number of C atoms and/or optionally substituted benzoyl groups are suitable. Multiply acylated alkylene diamines, particularly tetraacetylethyl ethylenediamine (TAED), acylated triazine derivatives, particularly 1,5-diacetyl-2,4-dioxohexahydro-1,3,5-triazine (DADHT), acylated glycolurils, particularly tetraacetyl glycoluril (TAGU), N-acylimides, particularly N-nonanoyl succinimide (NOSI), acylated phenol sulfonates, particularly n-nonanoyl- or isononanoyl oxybenzol sulfonate (n- or iso-NOBS), carboxylic acid anhydrides, particularly phthalic acid anhydride, acylated polyvalent alcohols, particularly triacetin, ethylene glycol diacetate, 2,5-diaceloxy-2,5-dihydrofuran and enol esters, as well as acylated sorbitol and mannitol and their described mixtures (SORMAN), acylated sugar derivatives, particularly pentaacetyl glucose (PAG), pentaacetyl fructose, tetraacetyl xylose, and octaacetyl lactose, as well as acylated, optionally N-acylated glucamine and gluconolactone, and/odes N-acylierte lactams, for example N-benzoyl caprolactam. The hydrophilically substituted acyl acetates and the acyl lactams are also preferably used. Combinations of conventional bleach activators can also be used. Such bleach activators, particularly in the presence of the abovementioned hydrogen peroxide-yielding bleaching agents, can be contained in the usual quantity range, preferably in quantities from about 0.5 wt % to about 10 wt %, particularly about 1 wt % to about 8 wt %, with respect to the overall agent, but are preferably omitted entirely when percarbonflic acid is used as the sole bleaching agent.

In addition to the conventional bleach activators or in their place, sulfonimines and/or bleach-boosting transition metal salts or transition metal complexes can also be contained as so-called bleach catalysts.

The organic solvents That can be used in the agents as contemplated herein besides water, particularly if they are present in liquid or pasty form, include alcohols with 1 to 4 C atoms, particularly methanol, ethanol, isopropanol, and tert,-butanol, diols with 2 to 4 C atoms, particularly ethylene glycol and propylene glycol, as well as mixtures thereof, and the ethers that can be derived from the cited classes of compounds. Such water-miscible solvents are preferably contained in the agents as contemplated herein in quantities of no more than 30 wt %, particularly from about 6 wt % to about 20 wt %.

To set a desired pH value that does not automatically result from the mixing of the other components, the agents as contemplated herein can contain system- and environmentally compatible acids, particularly citric acid, acetic acid, tartaric acid, malic acid, lactic acid, glycolic acid, succinic acid, glutaric acid, and/or adipic acid, but also mineral acids, particularly sulfuric acid, or bases, particularly ammonium or alkali hydroxides. Such pH regulators are preferably contained in the agents as contemplated herein in quantities of no more than about 20 wt %, particularly from about 1.2 wt % to about 17 wt %.

Anti-redeposition agents have the task of maintaining dirt loosened from the textile fiber suspended in the liquor. Water-soluble colloids mostly of an organic nature are suitable for this purpose—for example, starch, lime, gelatins, salts of ether carboxylic acids or ether sulfonic acids of starch or of cellulose, or salts of acidic sulfuric acid esters of cellulose or starch. Water-soluble, polyamides containing acidic groups are also suitable for this purpose. Moreover, starch derivatives other than those mentioned above, such as aldehyde starches, for example, can be used. Preferably, cellulose ethers such as carboxymethyl cellulose (Na salt), methyl cellulose, hydroxyalkyl cellulose, and mixed ethers such as methylhydroxyethyl cellulose, methylhydroxypropyl cellulose and mixtures thereof, for example, are used in quantities from about 0.1 to about 5 wt % with respect to the agent.

As optical brighteners, textile detergents as contemplated herein can contain derivatives of diaminostilbene disulfonic acid and alkali metal salts thereof, although they are preferably free of optical brighteners when for use as color detergents. The following are suitable, for example: salts of 4,4′-bis(2-anilino-4-morpholino-1,3,5-triazinyl-6-amino)stilbene-2,2′-disulfonic acid or similarly constructed compounds, which, instead of the morpholino group, bear a diethanolamine group, a methylamino group, an anilino group, or a 2-methoxyethylamino group. Furthermore, brighteners of the type of the substituted diphenyl styryls can be present, for example the alkali salts of 4,4′-bis(2-sulfostyryl)-diphenyls, 4,4′-bis(4-chloro-3-sulfostyryl)-diphenyls, or 4-(4-chlorostyryl)-4′-(2-sulfostyryl)-diphenyls. Mixtures of the abovementioned optical brighteners can also be used.

Particularly when used in mechanical processes, it can be advantageous to add common foam inhibitors to the agents. Soaps of a natural or synthetic origin are preferred as foam inhibitors, for example, which have a high proportion of C18-C24 fatty acids. Examples of suitable non-surfactant-like foam inhibitors are organopolysiloxanes and mixtures thereof with microfine, optionally silanized silicic acid as well as paraffins, waxes, microcrystalline waxes and mixtures thereof with silanized silicic acid or bis-fatty acid alkylene diamides. Mixtures of different foam inhibitors are also advantageously used, for example those of silicones, paraffins, or waxes. Preferably, the foam inhibitors, particularly silicone- or paraffin-containing foam inhibitors, are bound to a granular carrier substance that is soluble and/or dispersible in water. Mixtures of paraffins and bis-stearyl ethylenediamide are particularly preferred.

In other embodiments of agents as contemplated herein, particularly detergents or cleaning agents, the proteases are combined with one or more of the following ingredients, for example: nonionic, anionic, and/or cationic surfactants, (optionally other) bleaching agents, bleach activators, bleach catalysts, builders and/or cobuilders, acids, alkaline substances, hydrotropes, solvents, thickeners, sequestering agents, electrolytes, optical brighteners, anti-redeposition agents, corrosion inhibitors, particularly silver anti-tarnishing agents (silver corrosion inhibitors), disintegration aids, soil-release agents, color transfer inhibitors, foam inhibitors, abrasives, colorants, fragrances, perfumes, antimicrobial agents, UV protectants and/or absorbers, antistatic agents, pearlescent agents and skin protectants, enzymes such as proteases, amylases, cellulases, hemicellulases, mannanases, tannases, xylanases, xanthanases, β-glucosidases, carrageenases, oxidases, oxidoreductases, pectin-degrading enzymes or a lipase, stabilizers, particularly enzyme stabilizers, and other components that are known from the prior art. In another embodiment as contemplated herein, an agent is therefore characterized in that it contains at least one other component selected from the group consisting of surfactants, builders, acids, alkaline substances, hydrotropes, solvents, thickeners, bleaching agents, colorants, perfumes, corrosion inhibitors, sequestering agents, electrolytes, optical brighteners, anti-redeposition agents, silver corrosion inhibitors, color transfer inhibitors, foam inhibitors, disintegration aids, abrasives, UV absorbers, solvents, antistatic agents, pearlescent agents, and skin protectants.

The ingredients to be selected as well as the conditions in which the agent is used, such as temperature, pH value, ionic strength, redox ratios, for example, or mechanical influences, should be optimized for the respective cleaning problem. For instance, common temperatures for detergents and cleaning agents lie in ranges from about 10° C. for manual agents to about 40° C. and about 60° C. and up to about 95° C. for agents for machines or for technical applications. Since the temperature is usually infinitely adjustable in modern washing machines and dishwashers, all intermediate temperature levels are also included. The ingredients of the respective agent are preferably coordinated with one another. Synergies with respect to the cleaning performance are preferred. Synergies that result from the coaction of the two proteases in the agent as contemplated herein with at least one or more other ingredients are especially preferred in this regard. In another preferred embodiment, these synergies are present in a temperature range between about 20° C. and about 60° C., since the proteases contained in the agents as contemplated herein are also active in this temperature range.

In another embodiment, an agent as contemplated herein, particularly a detergent or cleaning agent, further comprises
about 5 wt % to about 70 wt %, particularly about 5 wt % to about 30 wt % surfactants and/or
about 10 wt % to about 65 wt %, particularly about 12 wt % to about 60 wt % water-soluble or water-dispersible inorganic builder material and/or
about 0.5 wt % to about 10 wt %, particularly about 1 wt % to about 8 wt % water-soluble organic builder substances and/or
about 0.01 to about 15 wt % solid inorganic and/or organic acids and/or acidic salts and/or
about 0.01 to about 5 wt % complexing agents for heavy metals and/or
about 0.01 to about 5 wt % anti-redeposition agent and/or
about 0.01 to about 5 wt % color transfer inhibitor and/or
about 0.01 to about 5 wt % foam inhibitor.

Optionally, the agent can further comprise optical brighteners, preferably from about 0.01 to about 5 wt %.

The manufacture of solid agents as contemplated herein does not pose any difficulty and can be carried out in a known manner, for example by spray-drying or granulation, with enzymes and any other thermally sensitive ingredients such as bleaching agents, for example, being added later separately. For the manufacture of agents as contemplated herein having a high bulk density, particularly in the range from about 650 g/l to about 950 g/l, a method having an extrusion step is preferred.

For the manufacture of agents as contemplated herein in tablet form, which can be single-phase or multiphase, single-colored or multicolored, and particularly consist of one or more layers, particularly of two layers, the procedure is preferably such that all of the components—optionally those of each layer—are mixed together in a mixer and the mixture is pressed using conventional tablet presses, for example eccentric presses or rotary presses, with pressing forces in the range from about about 50 to about 100 kN, preferably from about 60 to about 70 kN. Particularly in the case of multilayer tablets, it can be advantageous if at least one layer is prepressed. This is preferably done using pressing forces between about 5 and about 20 kN, particularly from about 10 to about 15 kN. In this way, tablets are obtained that are fracture-resistant but nonetheless sufficiently fast-dissolving under application conditions and have breaking and flexural strengths of normally from about 100 to about 200 N, but preferably over about 150 N. Preferably, a tablet manufactured in this way has a weight from about 10 g to about 50 g, particularly from about 15 g to about 40 g. The three-dimensional shape of the tablets can be freely determined and can be round, oval-shaped, or angular, with intermediate forms also being possible. Corners and edges are advantageously rounded off. Round tablets preferably have a diameter from about 30 mm to about 40 mm. The size particularly of angular or cuboid-shaped tablets, which are introduced predominantly via the dosing device of a dishwasher, for example, is dependent on the geometry and volume of this dosing device. For example, preferred embodiments have a base area of (about 20 to about 30 mm)×(about 34 to about 40 mm), particularly of about 26×36 mm or about 24×38 mm.

Liquid or pasty agents as contemplated herein in the form of solutions containing common solvents are generally manufactured simply by mixing the ingredients, which can be introduced into an automatic mixer in substance or as a solution. Embodiments of the present disclosure therefore include all such solid, powdered, liquid, gel-type, or pasty pharmaceutical forms of the agents, which can optionally also consist of several phases and be present in compressed or non-compressed form. Agents that are characterized in that they are present as a single-component system therefore represent another embodiment as contemplated herein.

Such agents preferably consist of one phase. As will readily be understood, however, agents as contemplated herein can also consist of several phases. In another embodiment as contemplated herein, the detergent or cleaning agent is therefore characterized in That it is divided into several components.

The solid pharmaceutical forms as contemplated herein therefore also include extrudates, granulates, tablets, or pouches, which can be present both in bulk containers and in single-portion packaging. Alternatively, the agent is present as a bulk powder, particularly with a bulk density from about 300 g/l to about 1200 g/l, particularly about 500 g/l to about 900 g/l or about 600 g/l to 850 g/about.

In another embodiment as contemplated herein, the agent, particularly the detergent or cleaning agent, is present in liquid, gel-type, or pasty form, particularly in the form of a non-aqueous liquid detergent or a non-aqueous paste or in the form of an aqueous liquid detergent or water-containing paste.

The agent as contemplated herein, particularly detergent or cleaning agent, can be packaged in a container, preferably an air-permeable container, from which it is released shortly before use or during the washing process. In particular, at least one, preferably both of the proteases contained in the agent and/or other ingredients of the agent can also be coated with a substance that is Impermeable for the enzyme(s) at room temperature or in the absence of water and becomes permeable for the enzyme(s) under the application conditions of the agent. Such an embodiment as contemplated herein is Therefore characterized in that at least one protease is coated with a substance That is impermeable for the protease at room temperature or in the absence of water.

Agents as contemplated herein can contain exclusively the at least two proteases described above. Alternatively, they can also contain other proteases or other enzymes in a concentration that is expedient for the effectiveness of the agent. Agents that further comprise one or more other enzymes therefore represent another object of the disclosure, with all enzymes established for this purpose in the prior art being usable in principle. AH enzymes that can have catalytic activity in the agent as contemplated herein, particularly proteases, amylases, cellulases, hemicellulases, mannanases, tannases, xylanases, xanthanases, β-glucosidasen, carrageenases, oxidases, oxidoreductases, pectin-degrading enzymes (pectinases) or lipases, as well as preferably mixtures thereof, can be preferably used as other enzymes. In principle, these enzymes are of natural origin; starting from the natural molecules, improved variants for use in detergents and cleaning agents are available which are preferably used accordingly. Agents as contemplated herein preferably contain enzymes in total quantities from about 1×10⁻⁸ to about 5 percent by weight with respect to active protein. The enzymes are preferably contained in agents as contemplated herein in quantities from about 0.00001 to about 5 wt %, more preferably from about 0.0001 to about 2.5 wt %, even more preferably from about 0.0001 to about 1 wt %, and especially preferably from about 0.0001 to about 0.072 wt %, it being possible for each enzyme contained to be present in the cited proportions.

The protein concentration can be determined with the aid of known methods, for example the BCA method (bichinchoninic acid; 2,2′-bichinolyl-4,4′-dicarboxylic acid) or the Biuret method (A. G. Gornall, C. S. Bardawill and M. M. David, J. Biol. Chem., 177 (1948), pp. 751-766).

Especially preferably, the other enzymes support the effect of the agent, for example the cleaning performance of a detergent or cleaning agent, in relation to specific contaminants or stains. Especially preferably, the enzymes exhibit synergistic effects with regard to their effect on specific contaminants or stains, that is, the enzymes contained in the agent composition support one another mutually in their cleaning performance. Synergistic effects can occur not only between different enzymes, but also between one or more enzymes and other ingredients of the agent as contemplated herein. In another preferred embodiment as contemplated herein, the agent is therefore characterized in that it contains at least one other enzyme that is a protease, amylase, cellulase, hemicellulase, mannanase, tannase, xylanase, xanthanase, β-glucosidase, carrageenase, oxidase, oxidoreductase, pectin-degrading enzyme, or a lipase.

In comparing the performance of two enzymes, a distinction must be made between protein-equivalent and activity-equivalent use. Particularly in the case of preparations that are obtained by genetic engineering and are largely free of side activity, protein-equivalent use is called for. After all, it is thus possible to determine whether the same quantities of protein—for example as a measure for the yield of fermentative production—lead to comparable results. If the respective ratios of active substance to total protein (the values for the specific activity) diverge from one another, then an activity-equivalent comparison is recommended, because the respective enzymatic characteristics are compared in that way. In general, a low specific activity can be compensated for through the addition of a greater quantity of protein. In the final analysis, this is an economic consideration.

The protease activity in such agents can be determined according to the method described in Tensile [Surfactants], Volume 7 (1970), pp. 125-132, It is indicated accordingly in PU (protease units).

The enzymes used in agents as contemplated herein originate either from microorganisms—for example, from the genera Bacillus, Streptomyces, Humicola, or Pseudomonas—and/or they are produced from suitable microorganisms using inherently known methods—for example, by using transgenic expression hosts of the genera Bacillus or using filamentous fungi.

The use of an above-described agent as contemplated herein for the removal of protease-sensitive contaminants on textiles or hard surfaces, i.e., for cleaning textiles or hard surfaces, represents a distinct object of the disclosure,

After all, particularly in light of the above-described characteristics, protease combinations as contemplated herein can be used to remove protein-containing contaminants from textiles or from hard surfaces. Hand-washing, the manual removal of stains from textiles or from hard surfaces, or use in conjunction with a mechanical method represent embodiments.

In one preferred embodiment of this use, the respective agents as contemplated herein, preferably detergents or cleaning agents, are provided in one of the embodiments described above.

Another distinct object of the disclosure is represented by methods for cleaning textiles or hard surfaces in which an agent as contemplated herein is used in at least one of the method steps. The method for cleaning textiles or hard surfaces is therefore characterized in that an agent as contemplated herein is used in at least one method step.

These include both manual and mechanical methods, with mechanical methods being preferred due to their precise controllability with respect to the quantities used and treatment times, for example.

Methods for cleaning textiles are generally characterized in that various substances that are active in detergency are applied to the article to be cleaned in several method steps and washed off after the treatment time, or that the article to be cleaned is otherwise treated with a detergent or a solution of this agent. The same applies to methods for cleaning all materials other than textiles, which are grouped together under the term “hard surfaces,” All conceivable washing or cleaning methods can be supplemented with an agent as contemplated herein in at least one of the method steps, upon which they constitute embodiments of the present disclosure.

Since preferred inventive combinations of proteases already naturally possess protein-degrading activity and exhibit it even in media that do not otherwise have any cleaning power, such as in mere buffers, for example, a single sub-step of such a method for the cleaning, particularly for the mechanical cleaning, of textiles or hard surfaces can consist in the fact that, as desired, an inventive mixture of enzymes is applied in addition to stabilizing compounds, salts, or buffer substances as the sole component that is active in detergency. This represents an especially simplified embodiment of the present disclosure.

Another object of the disclosure is represented by methods for cleaning textiles or hard surfaces that are characterized in that a first protease and at least one second protease are proteolytically active in at least one method step, wherein
a) the first protease comprises an amino acid sequence that is at least about 80% identical and, in order of increasing preference, at least about 82.5%, about 85%, about 87.5%, about 90%, about 91%, about 92%, about 93%, about 94%, about 95%, about 96%, about 97%, about 98%, about 99%, about 99.5% identical, and very especially preferably 100% identical to the amino acid sequence indicated in SEQ ID NO.1 and
b) the second protease is any protease that is different from the first protease, preferably selected from among proteases comprising an amino acid sequence that is at least about 80% and, in order of increasing preference, at least about 82.5%, about 85%, about 87.5%, about 90%, about 91%, about 92%, about 93%, about 94%, about 95%, about 96%, about 97%, about 98%, about 99%, about 99.5%, and very especially preferably 100% identical to one of the amino acid sequences indicated in SEQ ID NO.2 to SEQ ID NO.7, particularly from among proteases comprising an amino acid sequence that is at least about 80% identical and, in order of increasing preference, at least about 82.5%, about 85%, about 87.5%, about 90%, about 91%, about 92%, about 93%, about 94%, about 95%, about 96%, about 97%, about 98%, about 99%, about 99.5% identical, and very especially preferably 100% identical to one of the amino acid sequences indicated in SEQ ID NO.6 or SEQ ID NO.7, and
c) the optionally present other proteases can be any proteases that are different from the first and the second protease but are preferably selected from among proteases comprising an amino acid sequence that is at least about 80% and, in order of increasing preference, at least about 82.5%, about 85%, about 87.5%, about 90%, about 91%, about 92%. about 93%, about 94%, about 95%, about 96%, about 97%, about 93%, about 99%, about 99.5%, and very especially preferably 100% identical to one of the amino acid sequences indicated in SEQ ID NO.2 to SEQ ID NO.7.

The proteases are preferably used in a quantity from about 40 pg to about 4 g, preferably from about 50 μg to about 3 g, especially preferably from about 100 μg to about 2 g, and very especially preferably from about 200 μg to about 1 g per application. The first protease, the second protease, and the optionally present other proteases are included in the indicated quantities.

Other embodiments of this object of the disclosure are represented by methods for the treatment of textile raw materials or for textile care, in which an inventive combination of proteases is active in at least one of the method steps.

Among these, methods for textile raw materials, fibers, or textiles with natural components are preferred, and those with wool or silk are very especially preferred.

For example, these can be methods in which materials are prepared for processing into textiles, for instance for antifelting, or they can be methods which add a nurturing component to the cleaning of worn textiles. Due to the above-described effect of proteases on natural, protein-containing raw materials, in preferred embodiments, they are methods for the treatment of textile raw materials, fibers, or textiles with natural components, particularly with wool or silk.

Another distinct object of the disclosure is represented by the use of a first protease and at least one second protease for cleaning textiles or hard surfaces, characterized in that
d) the first protease comprises an amino acid sequence that is at least about 80% identical and, in order of increasing preference, at least about 82.5%, about 85%, about 87.5%, about 90%, about 91%, about 92%, about 93%, about 94%, about 95%, about 96%, about 97%, about 98%, about 99%, about 99.5% identical, and very especially preferably 100% identical to the amino acid sequence indicated in SEQ ID NO.1 and
e) the second protease is any protease that is different from the first protease, preferably selected from among proteases comprising an amino acid sequence that is at least about 80% and, in order of increasing preference, at least about 82.5%, about 85%, about 87.5%, about 90%, about 91%, about 92%, about 93%, about 94%, about 95%, about 96%, about 97%, about 98%, about 99%, about 99.5%, and very especially preferably 100% identical to one of the amino acid sequences indicated in SEQ ID NO.2 to SEQ ID NO.7, particularly from among proteases comprising an amino acid sequence that is at least about 80% identical and, in order of increasing preference, at least about 82.5%, about 85%, about 87.5%, about 90%, about 91%, about 92%, about 93%, about 94%, about 95%, about 96%, about 97%, about 98%, about 99%, about 99.5% identical, and very especially preferably 100% identical to one of the amino acid sequences indicated in SEQ ID NO.6 or SEQ ID NO.7, and
f) the optionally present other proteases can be any proteases that are different from the first and the second protease but are preferably selected from among proteases comprising an amino acid sequence that is at least about 80% and, in order of increasing preference, at least about 82.5%, about 85%, about 87.5%, about 90%, about 91%, about 92%, about 93%, about 94%, about 95%, about 96%, about 97%, about 98%, about 99%, about 99.5%, and very especially preferably 100% identical to one of the amino acid sequences indicated in SEQ ID NO.2 to SEQ ID NO.7.

After all, particularly in light of the above-described characteristics, protease combinations as contemplated herein can be used to remove protein-containing contaminants from textiles or from hard surfaces. Hand-washing, the manual removal of stains from textiles or from hard surfaces, or use in conjunction with a mechanical method represent embodiments. In a preferred embodiment of this use, the respective protease combinations as contemplated herein are provided according to one of the formulations described above.

Another object of the present disclosure is the use of a protease which comprises an amino acid sequence that is at least about 80% identical and, in order of increasing preference, at least about 82,5%, about 85%, about 87.5%, about 90%, about 91%, about 92%, about 93%, about 94%, about 95%, about 96%, about 97%, about 98%, about 99%, about 99.5% identical, and very especially preferably 100% identical to the amino acid sequence indicated in SEQ ID NO.1 as a booster for improving the proteolytic effect of other proteases or protease mixtures.

All facts, subject matter and embodiments that are described for detergents or cleaning agents as contemplated herein are applicable to the cited uses as well. Reference is therefore made here expressly to the disclosure in the corresponding location, with this disclosure also applying to the abovementioned uses as contemplated herein. The following examples explain the disclosure without limiting it: Example 1: Synergistic performance of proteases according to SEQ ID NO.1 and SEQ ID NO.6.

Four experiments were conducted under the following conditions:
1. Test contaminants used:
   Blood-milk/India ink on cotton: Product no. C5 from CFT B. V. Viaardingen, Holland
   Blood on cotton: Product no. CS1 from OFT B. V. Viaardingen, Holland
   Blood-milk/India ink on cotton: Product no. C3 from OFT B. V. Viaardingen, Holland
   Whole egg/India ink on cotton: Product no. CS37 from OFT B. V. Viaardingen, Holland
   Equest Napolitana Tomato Puree, available from Warwick Equest Ltd Unit 55, Consett Business Park, Consett, County Durham,
   Equest Grass & Mud, available from Warwick Equest Ltd Unit 55, Consett Business Park, Consett, County Durham,
   Equest French's Squeezy Mustard, available from Warwick Equest Ltd Unit 55, Consett Business Park, Consett, County Durham,

The washing tests were carried out in a Miele washing machine (Miele Novotronic W308) with 3.5 kg of ballast laundry.

Washing was performed a total of six times.

The liquor ratio was 1:4.

Dosing was as follows: 4 g of the agent/1000 ml.

The water hardness was 16 dH.

Washing was performed at two different temperatures:
g) at a washing temperature of 40° C. (program 6) for 60 minutes
h) at a washing temperature of 20° C. (program 30) for 60 minutes

Individual proteases and protease mixtures were worked into a Western European premium detergent having the following composition (ingredients in percent by weight, each with respect to the overall agent): 0.3-0.5% xanthan, 0.2-0.4% anti-foaming agent, 6-7% glycerin, 0.3-0.5% ethanol, 4-7% FAEOS (fatty alcohol ether sulfate), 24-28% nonionic surfactants, 1% boric acid, 1-2% sodium citrate (dihydrate), 2-4% soda, 14-16% coconut fatty acids, 0.5% HEDP (1-hydroxyethane-(1.1-diphosphonic acid)), 0-0.4% PVP (polyvinyl pyrrolidone), 0-0.05% optical brightener, 0-0.001% colorant, remainder demineralized water.

The total quantity of protease contained in one washload was 25 mg when only one protease according to SEQ ID NO.1, 6, or 7 was used. When a protease pair was used, 12.5 mg of the protease according to SEQ ID NO.1 and 25 mg of the protease according to SEQ ID NO.6 or SEQ ID NO.7 were added per washload.

The following tables show the relative proteolytic activity of a respective pair of proteases or of a pair of one protease and a protease mixture.
“nd” stands for “not determined”
I. Protease (SEQ ID NO.1) reduces contaminants mostly to a lesser extent than protease (SEQ ID NO.6) at a washing temperature of 20° C.:

| Contaminant | SEQ 6-SEQ 1 |
|---|---|
| CFT C05 | −8.1 |
| CFT C03 | −9.2 |
| CFT CS01 | nd |
| CFT CS37 | −4.4 |
| Equest Napolitana Tomato Puree | nd |
| Equest Grass & Mud | −0.5 |
| Equest French's Squeezy Mustard | 0.4 |

II. Protease (SEQ ID NO.1) reduces contaminants mostly to a lesser extent than protease (SEQ ID NO.7) at a washing temperature of 20° C.:

| Contaminant | SEQ 7-SEQ 1 |
|---|---|
| CFT C05 | −6.0 |
| CFT C03 | −8.2 |
| CFT CS01 | −0.8 |
| CFT CS37 | −4.8 |
| Equest Napolitana Tomato Puree | −0.8 |
| Equest Grass & Mud | nd |
| Equest French's Squeezy Mustard | nd |

III. An inventive mixture of protease (SEQ ID NO.1) and protease (SEQ ID NO.6) reduces contaminants mostly to a greater extent than protease (SEQ ID NO.6) alone at a washing temperature of 20° C.:

| Contaminant | SEQ 6-SEQ 6/SEQ 1 |
|---|---|
| CFT C05 | 0.8 |
| CFT C03 | 0.6 |
| CFT CS01 | nd |
| CFT CS37 | 1.5 |
| Equest Napolitana Tomato Puree | nd |
| Equest Grass & Mud | 1.0 |
| Equest French's Squeezy Mustard | 0.8 |

An inventive mixture of protease (SEQ ID NO.7) and protease (SEQ ID NO.1) reduces contaminants mostly to a greater extent than protease (SEQ ID NO.7) alone at a washing temperature of 20° C.:

| Contaminant | SEQ 7-SEQ 7/SEQ 1 |
|---|---|
| CFT C05 | 0.4 |
| CFT C03 | 1.4 |
| CFT CS01 | 2.2 |
| CFT CS37 | 1.9 |
| Equest Napolitana Tomato Puree | 1.9 |
| Equest Grass & Mud | nd |
| Equest French's Squeezy Mustard | nd |

Experiments III and IV document the superior synergistic effect of an agent as contemplated herein and of an enzyme mixture as contemplated herein, respectively.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein, it being understood that various changes may be made in the function and arrangement of elements described in an exemplary embodiment without departing from the scope of the various embodiments as set forth in the appended claims.

1. An agent, comprising a first protease and at least one second protease, wherein:
the first protease comprises an amino acid sequence that is at least about 80% identical to the amino acid sequence indicated in SEQ ID NO.1; and
the second protease is any protease that is different from the first protease.

2. The agent as set forth in claim 1, wherein the second protease is selected from among proteases comprising an amino acid sequence that is at least about 80% identical to one of the amino acid sequences indicated in SEQ ID NO.2 to SEQ ID NO.7.

3. The agent as set forth in claim 1, wherein, in addition to the first protease and the second protease, the agent comprises one or more other proteases.

4. The agent as set forth in claim 1, wherein the agent comprises the at least two proteases in total quantities from about 0.05 to about 5 wt % with respect to the total quantity of the agent.

5. The agent as set forth in claim 1, wherein the agent comprises the at least two proteases in total quantities from about 0.05 to about 15 wt % with respect to active protein.

6. The agent as set forth claim 1, further comprising at least one other component selected from the group of surfactants, builders, acids, alkaline substances, hydrotropes, solvents, thickeners, bleaching agents, colorants, perfumes, corrosion inhibitors, sequestering agents, electrolytes, optical brighteners, anti-redeposition agents, silver corrosion inhibitors, color transfer inhibitors, foam inhibitors, disintegration aids, abrasives, UV absorbers, solvents, antistatic agents, pearlescent agents, and skin protectants.

7. (canceled)

8. A method for cleaning textiles or hard surfaces, comprising:
activating proteolytically a first protease and at least one second protease;
wherein:
the first protease comprises an amino acid sequence that is at least about 80% identical to the amino acid sequence indicated in SEQ ID NO.1; and
the second protease is any protease that is different from the first protease.

9. The agent as set forth in claim 1 utilized for cleaning textiles or hard surfaces.

10. A protease comprising an amino acid sequence that is at least about 80% identical to the amino acid sequence indicated in SEQ ID NO.1 utilized as a booster for improving the proteolytic effect of other proteases or protease mixtures.

11. The agent as set forth in claim 1, wherein the second protease is selected from among proteases comprising an amino acid sequence that is at least about 90% identical to one of the amino acid sequences indicated in SEQ ID NO.2 to SEQ ID NO.7.

12. The agent as set forth in claim 1, wherein the second protease is selected from among proteases comprising an amino acid sequence that is at least about 95% identical to one of the amino acid sequences indicated in SEQ ID NO.2 to SEQ ID NO.7.

13. The agent as set forth in claim 1, wherein the second protease is selected from among proteases comprising an amino acid sequence that is at least about 80% identical to one of the amino acid sequences indicated in SEQ ID NO.6 or SEQ ID NO.7.

14. The agent as set forth in claim 1, wherein the second protease is selected from among proteases comprising an amino acid sequence that is at least about 90% identical to one of the amino acid sequences indicated in SEQ ID NO.6 or SEQ ID NO.7.

15. The agent as set forth in claim 1, wherein the second protease is selected from among proteases comprising an amino acid sequence that is at least about 95% identical to one of the amino acid sequences indicated in SEQ ID NO.6 or SEQ ID NO.7.

16. The agent as set forth in claim 1, wherein, in addition to the first protease and the second protease, the agent comprises one or more other proteases selected from among proteases comprising an amino acid sequence that is at least 80% identical to one of the amino acid sequences indicated in SEQ ID NO.2 to SEQ ID NO.7.

17. The agent as set forth in claim 1, wherein, in addition to the first protease and the second protease, the agent comprises one or more other proteases selected from among proteases comprising an amino acid sequence that is at least 90% identical to one of the amino acid sequences indicated in SEQ ID NO.2 to SEQ ID NO.7.

18. The agent as set forth in claim 1, wherein, in addition to the first protease and the second protease, the agent comprises one or more other proteases selected from among proteases comprising an amino acid sequence that is at least 95% identical to one of the amino acid sequences indicated in SEQ ID NO.2 to SEQ ID NO.7.

19. The agent as set forth claim 1, wherein the agent comprises the at least two proteases in total quantities from about 0.05 to about 2 wt % with respect to the total quantity of the agent.

20. The agent as set forth in claim 1, wherein the agent comprises the at least two proteases in total quantities from about 0.05 to about 10 wt % with respect to active protein.

21. An agent, comprising: a first protease and at least one second protease; wherein:
the first protease comprises an amino acid sequence that is at least about 80% identical to the amino acid sequence indicated in SEQ ID NO.1; and
the second protease is selected from among proteases comprising an amino acid sequence that is at least about 80% identical to one of the amino acid sequences indicated in SEQ ID NO.2 to SEQ ID NO.7; and
wherein, in addition to the first protease and the second protease, the agent comprises one or more other proteases selected from among proteases comprising an amino acid sequence that is at least 80% identical to one of the amino acid sequences indicated in SEQ ID NO.2 to SEQ ID NO.7.


SEQUENCE LISTING (7 sequences)

SEQ ID NO: 1 - protein, 274 aa, Bacillus Licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 2 - protein, 379 aa, Bacillus Licheniformis
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGTYSGIVSGIEWATTNGMDVINMSLGGPSGSTAMKQAVDNAYARGVVVVAAAGNSGSSGNTNTIGYPAKYDSVIAVGAVDSNSNRASFSSVGAELEVMAPGAGVYSTYPTSTYATLNGTSMASPHVAGAAALILSKHPNLSASQVRNRLSSTATYLGSSFYYGKGLINVEAAAQ

SEQ ID NO: 3 - protein, 275 aa, Bacillus amyloliquefaciens [sequence omitted]

SEQ ID NO: 4 - protein, 387 aa, Bacillus licheniformis
SEQENZASMMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 5 - protein, 379 aa, Bacillus licheniformis; identical to SEQ ID NO: 2

SEQ ID NO: 6 - protein, 269 aa, Bacillus Lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 7 - protein, 275 aa, Bacillus amyloliquefaciens
AQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGALNGTSMASPHVAGAAALILSKHPNWTNTQVRSSLENTTTKLGDSFYYGKGLINVQAAAQ